(19) United States
(12) Patent Application Publication
(10) Pub. No.: US 2019/0000911 A1
(43) Pub. Date: Jan. 3, 2019

(54) HERBAL COMPOSITION FOR HEALING OF WOUND, CUTS AND BURNS AND A METHOD OF SYNTHESIZING THEREOF

(71) Applicant: Fariba Sepahvand, Khorramabad (IR)

(72) Inventor: Fariba Sepahvand, Khorramabad (IR)

(21) Appl. No.: 16/127,263

(22) Filed: Sep. 11, 2018

(51) Int. Cl. A61K 36/884; A61K 9/06; A61K 47/06; A61P 17/02

(52) U.S. Cl. CPC A61K 36/884; A61K 9/06; A61K 47/06; A61P 17/02; A61K 2236/51; A61K 2236/17; A61K 2236/15; A61K 2236/333; A61K 2236/13

(57) ABSTRACT

The embodiments herein disclose a method for synthesizing an ointment from water plantain for the wounds, cuts and burnt tissues. The water plantain plant sample is collected and subjected for botanical analysis. The water plantain plant sample are pre-treated and dried. The dried water plantain plant samples are grinded to a powder. The water plantain plant powder is subjected to maceration/soaking for obtaining hydroalcoholic extract. A total of 5 litres of hydroalcoholic solvent extract comprising the essential nutrients and components of water plantain plant is obtained. A predetermined amount of the hydroalcoholic solvent extract is condensed in a rotary evaporator. The water plantain plant powder and hydroalcoholic solvent extract are tested for blood clotting. The hydroalcoholic solvent extract is mixed with Vaseline to obtain an ointment. The ointment is tested on rat wounds to find that the wound is healed in 15 days.

### BACKGROUND

### Technical Field

The embodiments herein are generally related to a field of health care and medical formulation/composition. The embodiments herein are particularly related to an herbal composition for an oral application for the treatment of tissues with cuts, burns and wounds. The embodiment herein are more particularly related to an herbal composition for an ointment for application over tissues with cuts, burns and wounds, and a method of synthesizing the same.

### Description of the Related Art

Burns, cuts and wounds are caused by disruption of the cellular and anatomical continuity of an organism or the body part. The burns, cuts and skin wounds are traumatic. The burns, cuts and wounds may get infected or give rise to medical issues. The burns and wounds are repaired by inflammation, proliferation of cells and remodeling.

The new tissue formation starts with reepithelialization and is followed by granulation tissue formation. Reepithelialization comprises macrophage accumulation, fibroblast growth, matrix decomposition and angiogenesis. The tissue repair is driven by growth factors and cytokines. Wound, cuts and burn healing is a physiologic process, but sometimes there is delay in wound healing. Delay in wound healing is attributed to immune disorders, diabetes, ischemia, venous stasis, malnutrition, metabolic deficiencies, burn, gunshot wounds and aging.

The chemical formulation has been reported for wound and burn healing. But chemical formulation must be used for longer duration and long period of time. The chemical formulation may lead to low frequency hypo-sensitivity reactions, toxicity and irritation upon application in the affected area. Further chemical formulation may appear to be resistant for plurality of strains of microorganisms.

The chemical based composition uses coagulation factors and tissue repair factors which are usually produced chemically. The course of treatment is also long. Further the chemical based compositions are costly.

Many plants and plant extracts are known in the traditional medicines which are used for the treatment of burns, cuts and wounds.

Water plantain is a perennial plan with a length up to one meter. The plant has broad leaves which are tapered or round at base. The water plantain belongs to the genus Alisma.

The water plantain plant is found throughout the world mainly near rivers and marsh lands. The rhizome of the water plantain is used in curing dropsy, gonorrhea, diabetes and kidney disease. The rhizome is astringent and increase milk secretion. The researches illustrate that the fruit have properties like rhizome. But fruits are known to make men sterile.

In traditional Chinese medicine, the water plantain plant is used to facilitate child delivery decreasing pain and accelerating childbirth. The leaves are used to cure leprosy. The roots are known to make eyes and ears sensitive. The water plantain is also used for weight loss and clear skin. It has been reported that the water plantain cures vitiligo.

The known methods for treating bleeding, wounds and cuts use coagulation factors and tissue repair factors. The coagulation factors and the tissue repair factors are synthesized artificially or chemical synthesis. The time period of treatment is long and the cost involved in using the artificially synthesized coagulation factors and tissue repair factors is more.

Water plantain is also known in treating bleeding patients, repairing tissues and burns as it comprises resin.

Hence there is a need for an herbal composition synthesized from water plantain comprising resin and fatty acids for targeting/healing wounds, cuts and burns. Also, there is a need for a simple method for extracting the herbal extract from water plantain.

The above-mentioned shortcomings, disadvantages and problems are addressed herein and which will be understood by reading and studying the following specification.

### OBJECTIVES OF THE EMBODIMENTS

The primary objective of the embodiment herein is to provide an herbal composition for targeting/healing wounds, cuts and burns.

Another object of the embodiment herein is to provide a simple method for synthesis of herbal extract composition from water plantain plant.

Yet another object of the embodiment herein is to provide an herbal extract composition from water plantain which is easy to apply on the wounds, cuts and burnt tissues.

Yet another object of the embodiment herein is to provide an herbal extract composition from water plantain plant which prevents pathogen growth and is an anti-bleeding.

Yet another object of the embodiment herein is to provide an herbal extract composition from water plantain which has no side effects on the healing of cuts, wounds and burns.

Yet another object of the embodiment herein is to provide an herbal extract composition from water plantain which has a low cost.

Yet another object of the embodiment herein is to provide an herbal extract composition from water plantain which heals the wounds, cuts and burnt tissues in short time period when compared to the existing artificially synthesized tissue regeneration agents and healing factors.

These objects and the other advantages of the embodiments herein will become readily apparent from the following detailed description taken in conjunction with the accompanying drawings.

### SUMMARY

The various embodiments herein provide an herbal composition for targeting/healing wounds, cuts and burns. Further a simple method is provided for synthesis of an herbal extract composition from water plantain plant. The herbal extract composition synthesized from water plantain is easily applied on the wounds, cuts and burnt tissues.

According to one embodiment herein, the method for extracting the hydro alcoholic solvent extract comprising nutrients and components of a water plantain plant for synthesizing an ointment for application on wounds, cuts and burnt tissues, comprises the following steps. A plurality of water plantain plant samples are generated. The plurality of water plantain plant samples are pre-treated. The plurality of water plantain plant samples are cleaned and dis-infected. The plurality of cleaned and disinfected water plantain plant samples are dried in open air and in normal atmospheric conditions. The plurality of water plantain plant samples are dried in a shade area or dark room without exposing to any light source. The dried water plantain plant samples are ground to obtain a powder using a grinding machine and a sieve. The water plantain plant powder is subjected for maceration/soaking in a solvent for obtaining hydro alcoholic extract for synthesizing an ointment. The predetermined amount of hydro alcoholic solvent extract is condensed by heating in a water bath of a rotary evaporator for a predetermined time period. The hydro alcoholic solvent extract is heated to separate the solvent and to obtain a concentrated hydro alcoholic solvent extract. The concentrated hydro alcoholic solvent extract is cooled. A plurality of nutrients and components present in the hydro alcoholic solvent extract obtained from the water plantain plant are analysed by Gas chromatography-mass spectrometry (GC-MS) machine. The hydro alcoholic solvent extract obtained from the water plantain plant and the powder of the water plantain plant are analysed for blood clotting blood/blood coagulating properties.

According to one embodiment herein, the step of pre-treating the water plantain plant sample comprises the following steps. The water plantain plant samples of genus Alisma are collected in the spring season. The collected water plantain plant samples are dried in open air, avoiding light and in normal atmospheric conditions. It is determined whether the collected water plantain plant samples belong to genus Alisma by testing the water plantain plant samples in a herbarium. The collected water plantain plant samples are confirmed to belong to genus Alisma.

According to one embodiment herein, the step of subjecting the water plantain plant powder for maceration/soaking in a solvent for obtaining hydro alcoholic extract for synthesizing an ointment, comprises the following steps. 677 grains of the water plantain plant powder is added in a percolator machine. 3500 cc of solvent is added in the percolator machine comprising water plantain plant powder. The solvent is 70% v/v ethanol. The water plantain plant powder with solvent is incubated in percolator machine for a predetermined time period. The predetermined time period is 72 hours.1800 cc of the solvent extract is collected after 72 hours by opening outlet of percolator machine. 2000 cc of solvent is added into the percolator machine comprising a mixture of solvent and water plantain plant powder. The solvent is the 70% v/v ethanol. The solvent, the mixture of solvent and water plantain plant powder are incubated in percolator machine for a predetermined time period. The predetermined time period is 48 hours. 1800 cc of the solvent extract is collected after 48 hours by opening outlet of percolator machine. 2000 cc of solvent (70%v/v ethanol) is added in the percolator machine comprising a mixture of solvent and water plantain plant powder. The solvent is the 70% v/v ethanol. The solvent, the mixture of solvent and water plantain plant powder is incubated in percolator machine for a predetermined period of time. The predetermined time period is 24 hours. 1400 cc of the solvent extract is collected after 24 hours by opening an outlet of percolator machine. A total of 5 litres (1800 cc+1800 cc+1400 cc) of hydro alcoholic solvent extract, comprising the essential nutrients and natural components of water plantain plant, is obtained.

According to one embodiment herein, the Gas chromatography-mass spectrometry (GC-MS) analysis of hydro alcoholic solvent extract obtained from the plurality of water plantain samples illustrates a presence of a plurality of nutrients and components. The plurality of nutrients and components are proteins, starch, resins, furfur aldehyde, aspargine, alkaloid, sugar, phytosterol, a plurality of fatty acids and a plurality of colour less solids.

According to one embodiment herein, the plurality of fatty acids are palmitic acid, stearic acid, linoleic acid and oleic acid.

According to one embodiment herein, the resin component in the hydro alcoholic solvent extract is a tissue regeneration agent. The resin provides nutrients required for a cell division. The resin promotes a tissue healing process.

According to one embodiment herein, the hydro alcoholic solvent extract at a plurality of concentration levels are tested for blood clotting/blood coagulation property. The plurality of concentration levels of the hydro alcohol solvent extract are 25 mg/L (λ), 50 mg/L (λ), 75 mg/L (λ) and 100 mg/L (λ). The hydro alcohol solvent extract with a concentration of 25 mg/L (λ) illustrate a time period of 4 minute 15 seconds for clotting 2 cc of blood when compared to hydro alcohol solvent extract with a concentration of the 50 mg/L (λ), 75 mg/L (λ) and 100 mg/L (λ)

According to one embodiment herein, the composition for application on wounds, cuts and burnt tissues, comprises a predetermined amount of hydro alcoholic solvent extract and a predetermined amount of petroleum jelly. The petroleum jelly is Vaseline. The hydro alcoholic solvent extract is obtained from the water plantain plant powder by a maceration/soaking method. A plurality of nutrients and components are present in the hydro alcoholic solvent extract obtained from water plantain.

According to one embodiment herein, the plurality of nutrients and components present in the hydro alcoholic solvent extract are a plurality of proteins, starch, resins, furfur aldehyde, asparagine, alkaloid, sugar, phytosterol, a plurality of fatty acids and a plurality of colour less solids.

According to one embodiment herein, the plurality of fatty acids are palmitic acid, stearic acid, linoleic acid and oleic acid.

According to one embodiment herein, the resin component in the hydro alcoholic solvent extract is a tissue regeneration agent. The resin provides nutrients required for a cell division. The resin promotes tissue healing.

According to one embodiment herein, the method synthesizing the herbal composition comprises the following steps. The water plantain plant is collected in spring season. The water plantain plant is air dried. The water plantain plant is identified and determined in an herbarium for confirming the botanical parameters.

According to one embodiment herein, the water plantain plant cleaned and disinfected. The water plantain plants are dried for one week in open air away from light and in normal atmospheric condition. The dried water plantain plants are grinded to obtain a powder using grinding machine and a sieve. A pre-determined amount (677 grains) of water plantain plant powder is subjecting to maceration/soaking method. 3500 cc of solvent (70% v/v ethanol) is added in a percolator machine comprising dried powder of water plantain plant. The percolator machine comprising 3500 cc of solvent and dried powder of water plantain plant is incubated for 72 hours. After 72 hours 1800 cc of solvent extract is collected from the percolator machine by opening the outlet. 2000 cc of solvent (70% v/v ethanol) is added to the percolator machine comprising solvent and water plantain plant powder. Incubating the percolator machine for 48 hours. After 48 hours 1800 cc of solvent extract is collected from the percolator machine. 2000 cc of solvent (70% v/v ethanol) is added to the percolator machine comprising solvent and water plantain plant powder. Incubating the percolator machine for 24 hours. After 24 hours 1400 cc of solvent extract is collected from the percolator machine. A total of 5 liters of hydroalcoholic solvent extract is obtained (1800 cc+1800 cc+1400 cc). The hydroalcoholic solvent extract comprises the essential nutrients and components of water plantain plant. The hydroalcoholic solvent extract is pulpous in nature or state, as the solvent extract comprises alcohol. A predetermined amount (297/7 grains) of the solvent extract is condensed by using a rotary evaporator to obtain a concentrated substance of hydroalcohol. The rotary evaporator comprises a water bath. The solvent extract is heated in the water bath, to separate the solvent. After heating the solvent extract for a predetermined time period, the solvent extract is cooled. The extract is pulpy in nature.

According to one embodiment herein, the ointment for treating burns, wounds and cuts in tissues comprises a predetermined amount concentrated substance of hydro alcohol/concentrated hydro alcoholic solvent extract and predetermined amount of Vaseline.

According to one embodiment herein, the percolator device is made of glass. The water plantain plant powder and the solvent (70% v/v ethanol) flows through the percolator device. The solvent flows in the percolator device without the intervention of the water plantain plant powder.

According to one embodiment herein, the chemical analysis of the water plantain solvent extract is done by Gas chromatography-mass spectrometry (GC-MS) machine. The water plantain solvent extract (hydroalcoholic solvent extract) comprises plurality of proteins, starch and resins. The GC-MS analysis illustrate presence of furfur aldehyde, asparagine, alkaloid, sugar, phytosterol, a combination of fatty acids and colorless solids.

The results obtained from the GC-MS analysis further illustrate the presence of resin and fatty acids mainly in the water plantain solvent extract (hydro alcoholic solvent extract). The resin component in the water plantain solvent extract (hydro alcoholic solvent extract) is the effective element of the solvent extract. The resin component repairs the tissues and stops bleeding. The fatty acids in the water plantain solvent extract are palmitic acid, stearic acid, linoleic acid and oleic acid. The fatty acids are the nutrient elements which aid in repairing the tissues. The GC-MS machine illustrates the presence of alkaloid and sugar in the water plantain solvent extract.

According to one embodiment herein, fatty acids in the water plantain solvent extract (hydro alcoholic solvent extract) are the nutrients and growth factors for healing the wounds, cuts and burnt tissues. The resin component in the water plantain solvent extract (hydro alcoholic solvent extract) is tissue regeneration agent. The resin provides environmental nutrients for cell division and tissue healing process.

According to one embodiment herein, the effect of the hydro alcoholic solvent extract of the water plantain solvent extract and the powder of the water plantain is determined on the blood coagulation. A predetermined amount hydro alcoholic solvent extract of water plantain is poured into the test pipes (test pipe 1, 2, 3 and 4) with the help of a pipette. The test pipes are covered and shaken for a predetermined time period. The hydro alcoholic solvent extract of the water plantain is dried in the test pipes. After drying the hydro alcoholic solvent extract of water plantain, 2 cc of blood sample is added into test pipes. The blood clotting time is measured in all the test pipes. The test pipe 5 is control pipe, as hydro alcoholic solvent extract of water plantain is not added in the test pipe. The test pipe 6 comprises the water plantain plant powder and blood sample.

According to one embodiment herein, the results illustrate that the test pipe 1 comprising less water plantain hydro alcoholic solvent extract takes less time for clotting blood sample when compared to the water plantain powder.

According to one embodiment herein, the ointment formulation comprises a predetermined amount of hydro alcoholic solvent extract of water plantain and a predetermined amount of Vaseline.

According to one embodiment herein, the ointment comprising hydro alcoholic solvent extract of water plantain and Vaseline is tested for treating burns and bleeding in rats. The skin wounds are created on the skin of a plurality of rats. The rats are anesthetized by intraperitoneal injection of ketamine xylazine.

The rat's nape is cleaned with a razor. A shape “O” is taken from the rat skin to get a plurality of wounded regions. The topical treatment of the skin wound is initiated on the first day. The topical treatment comprises application of the ointment comprising hydro alcoholic solvent extract of water plantain and Vaseline. The ointment comprising hydro alcoholic solvent extract of water plantain and Vaseline is a semisolid. The semisolid ointment comprising hydro alcoholic solvent extract of water plantain and vaseline is applied on the wounds using swaps twice a day. The day of creating a rat skin wound and starting the topical treatment is “zero-day”. During the different stages of topical treatment, the compliance with ethical issues is maintained and any form of physical abuse and non-essential methods to the rats are avoided. On the rat's body, a wound is considered as control wound. The control wound does not receive any topical treatment. The topical treatment of the rat skin wound is continued for two weeks. On the third day, the wound receiving the topical treatment illustrated a reduction in the diameter of the wound. On fourteenth day, a complete recovery of the wound receiving the topical treatment with the ointment comprising hydro alcoholic solvent extract of water plantain and Vaseline is observed.

According to one embodiment herein, the most effective nutrients/elements present in the water plantain solvent extract stops bleeding and promote healing of tissues. The nutrients/elements of water plantain solvent extract which stops bleeding and promote healing of tissues include resin, fatty acids, palmitic acid and stearic acid. The resin heals the wound faster. The resin heals the wound faster as the resin has adherence properties for the treatment of wounds and burns. The starch and the carbohydrate present in the water plantain solvent extract act a growth factor for tissue.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flowchart explaining a method for pre-treatment of the water plantain plant samples, according to one embodiment herein.

FIG. 2 illustrates a flowchart explaining a method for extracting the hydro alcoholic solvent extract comprising the essential nutrients and components of water plantain plant for synthesizing an ointment for treating the wounds, cuts and burnt tissues, according to one embodiment herein.

FIG. 3 illustrates a flow chart explaining a method for maceration/soaking for obtaining hydro alcoholic solvent extract for synthesizing an ointment, according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide an herbal composition for targeting/healing wounds, cuts and burns. Further a simple method for synthesis of herbal extract composition from water plantain plant is provided. The herbal extract composition synthesized from water plantain is easily applied on the wounds, cuts and burnt tissues.

According to one embodiment herein, the method for extracting the hydro alcoholic solvent extract comprising nutrients and components of a water plantain plant for synthesizing an ointment for application on wounds, cuts and burnt tissues, comprises the following steps. A plurality of water plantain plant samples are generated. The plurality of water plantain plant samples are pre-treated. The plurality of water plantain plant samples are cleaned and dis-infected. The plurality of cleaned and disinfected water plantain plant samples are dried in open air and in normal atmospheric conditions. The plurality of water plantain plant samples are dried in a shade area or dark room without exposing to any light source. The dried water plantain plant samples are ground to obtain a powder using a grinding machine and a sieve. The water plantain plant powder is subjected for maceration/soaking in a solvent for obtaining hydro alcoholic extract for synthesizing an ointment. The predetermined amount of hydro alcoholic solvent extract is condensed by heating in a water bath of a rotary evaporator for a predetermined time period. The hydro alcoholic solvent extract is heated to separate the solvent and to obtain a concentrated hydro alcoholic solvent extract. The concentrated hydro alcoholic solvent extract is cooled. A plurality of nutrients and components present in the hydro alcoholic solvent extract obtained from the water plantain plant are analysed by Gas chromatography-mass spectrometry (GC-MS) machine. The hydro alcoholic solvent extract obtained from the water plantain plant and the powder of the water plantain plant are analysed for blood clotting blood/blood coagulating properties.

According to one embodiment herein, the step of pre-treating the water plantain plant sample comprises the following steps. The water plantain plant samples of genus Alisma are collected in the spring season. The collected water plantain plant samples are dried in open air, avoiding light and in normal atmospheric conditions. It is determined whether the collected water plantain plant samples belong to genus Alisma by testing the water plantain plant samples in a herbarium. The collected water plantain plant samples are confirmed to belong to genus Alisma.

According to one embodiment herein, the step of subjecting the water plantain plant powder for maceration/soaking in a solvent for obtaining hydro alcoholic extract for synthesizing an ointment, comprises the following steps. 677 grains of the water plantain plant powder is added in a percolator machine. 3500 cc of solvent is added in the percolator machine comprising water plantain plant powder. The solvent is 70% v/v ethanol. The water plantain plant powder with solvent is incubated in percolator machine for a predetermined time period. The predetermined time period is 72 hours.1800 cc of the solvent extract is collected after 72 hours by opening outlet of percolator machine. 2000 cc of solvent is added into the percolator machine comprising a mixture of solvent and water plantain plant powder. The solvent is the 70% v/v ethanol. The solvent, the mixture of solvent and water plantain plant powder are incubated in percolator machine for a predetermined time period. The predetermined time period is 48 hours. 1800 cc of the solvent extract is collected after 48 hours by opening outlet of percolator machine. 2000 cc of solvent (70%v/v ethanol) is added in the percolator machine comprising a mixture of solvent and water plantain plant powder. The solvent is the 70% v/v ethanol. The solvent, the mixture of solvent and water plantain plant powder is incubated in percolator machine for a predetermined period of time. The predetermined time period is 24 hours. 1400 cc of the solvent extract is collected after 24 hours by opening an outlet of percolator machine. A total of 5 litres (1800 cc+1800 cc+1400 cc) of hydro alcoholic solvent extract, comprising the essential nutrients and natural components of water plantain plant, is obtained.

According to one embodiment herein, the Gas chromatography-mass spectrometry (GC-MS) analysis of hydro alcoholic solvent extract obtained from the plurality of water plantain samples illustrates a presence of a plurality of nutrients and components. The plurality of nutrients and components are proteins, starch, resins, furfur aldehyde, aspargine, alkaloid, sugar, phytosterol, a plurality of fatty acids and a plurality of colour less solids.

According to one embodiment herein, the plurality of fatty acids are palmitic acid, stearic acid, linoleic acid and oleic acid.

According to one embodiment herein, the resin component in the hydro alcoholic solvent extract is a tissue regeneration agent. The resin provides nutrients required for a cell division. The resin promotes a tissue healing process.

According to one embodiment herein, the hydro alcoholic solvent extract at a plurality of concentration levels are tested for blood clotting/blood coagulation property. The plurality of concentration levels of the hydro alcohol solvent extract are 25 mg/L (λ), 50 mg/L (λ), 75 mg/L (λ) and 100 mg/L (λ). The hydro alcohol solvent extract with a concentration of 25 mg/L (λ) illustrate a time period of 4 minute 15 seconds for clotting 2 cc of blood when compared to hydro alcohol solvent extract with a concentration of the 50 mg/L (λ), 75 mg/L (λ) and 100 mg/L (λ)

According to one embodiment herein, the composition for application on wounds, cuts and burnt tissues, comprises a predetermined amount of hydro alcoholic solvent extract and a predetermined amount of petroleum jelly. The petroleum jelly is Vaseline. The hydro alcoholic solvent extract is obtained from the water plantain plant powder by a maceration/soaking method. A plurality of nutrients and components are present in the hydro alcoholic solvent extract obtained from water plantain.

According to one embodiment herein, the plurality of nutrients and components present in the hydro alcoholic solvent extract are a plurality of proteins, starch, resins, furfur aldehyde, asparagine, alkaloid, sugar, phytosterol, a plurality of fatty acids and a plurality of colour less solids.

According to one embodiment herein, the plurality of fatty acids are palmitic acid, stearic acid, linoleic acid and oleic acid.

According to one embodiment herein, the resin component in the hydro alcoholic solvent extract is a tissue regeneration agent. The resin provides nutrients required for a cell division. The resin promotes tissue healing

According to one embodiment herein, the method synthesizing the herbal composition comprises the following steps. The water plantain plant is collected in spring season. The water plantain plant is air dried. The water plantain plant is identified and determined in an herbarium for confirming the botanical parameters. A herbarium code is generated and confirmation is obtained regarding the genus of water plantain i.e. Alisma.

According to one embodiment herein, the water plantain plant cleaned and disinfected. The water plantain plants are dried for one week in open air away from light and in normal atmospheric condition. The dried water plantain plants are grinded to obtain a powder using grinding machine and a sieve. A pre-determined amount (677 grains) of water plantain plant powder is subjecting to maceration/soaking method. 3500 cc of solvent (70% v/v ethanol) is added in a percolator machine comprising dried powder of water plantain plant. The percolator machine comprising 3500 cc of solvent and dried powder of water plantain plant is incubated for 72 hours. After 72 hours 1800 cc of solvent extract is collected from the percolator machine by opening the outlet. 2000 cc of solvent (70% v/v ethanol) is added to the percolator machine comprising solvent and water plantain plant powder. Incubating the percolator machine for 48 hours. After 48 hours 1800 cc of solvent extract is collected from the percolator machine. 2000 cc of solvent (70% v/v ethanol) is added to the percolator machine comprising solvent and water plantain plant powder. Incubating the percolator machine for 24 hours. After 24 hours 1400 cc of solvent extract is collected from the percolator machine. A total of 5 liters of hydroalcoholic solvent extract is obtained (1800 cc+1800 cc+1400 cc). The hydroalcoholic solvent extract comprises the essential nutrients and components of water plantain plant. The hydroalcoholic solvent extract is pulpous in nature or state, as the solvent extract comprises alcohol. A predetermined amount (297/7 grains) of the hydroalcoholic solvent extract is condensed by using a rotary evaporator to obtain a concentrated substance of hydroalcohol. The rotary evaporator comprises a water bath. The solvent extract is heated in the water bath, to separate the solvent. After heating the solvent extract for a predetermined time period, the solvent extract is cooled. The extract is pulpy in nature.

According to one embodiment herein, the ointment for treating burns, wounds and cuts in tissues comprises a predetermined amount concentrated substance of hydroalcohol/concentrated hydroalcoholic solvent extract and predetermined amount of Vaseline.

According to one embodiment herein, the percolator device is made of glass. The water plantain plant powder and the solvent (70% v/v ethanol) flows through the percolator device. The solvent flows in the percolator device without the intervention of the water plantain plant powder.

According to one embodiment herein, the chemical analysis of the water plantain solvent extract is done by Gas chromatography-mass spectrometry (GC-MS) machine. The water plantain solvent extract (hydroalcoholic solvent extract) comprises plurality of proteins, starch and resins. The GC-MS analysis illustrate presence of furfuraldehyde, aspargine, alkaloid, sugar, phytosterol, a combination of fatty acids and colorless solids.

The results obtained from the GC-MS analysis further illustrate the presence of resin and fatty acids mainly in the water plantain solvent extract (hydroalcoholic solvent extract). The resin component in the water plantain solvent extract (hydroalcoholic solvent extract) is the effective element of the solvent extract. The resin component repairs the tissues and stops bleeding. The fatty acids in the water plantain solvent extract are palmitic acid, stearic acid, linoleic acid and oleic acid. The fatty acids are the nutrient elements which aid in repairing tissues. The GC-MS machine illustrates the presence of alkaloid and sugar in the water plantain solvent extract.

According to one embodiment herein, fatty acids in the water plantain solvent extract (hydroalcoholic solvent extract) are the nutrients and growth factors for healing the wounds, cuts and burnt tissues. The resin component in the water plantain solvent extract (hydroalcoholic solvent extract) is tissue regeneration agent. The resin provides environmental nutrients for cell division and tissue healing process.

According to one embodiment herein, the effect of the hydroalcoholic solvent extract of the water plantain solvent extract and the powder of the water plantain is determined on the blood coagulation. A predetermined amount hydroalcoholic solvent extract of water plantain is poured into the test pipes (test pipe 1, 2, 3 and 4) with the help of a pipette. The test pipes are covered and shaken for a predetermined time period. The hydroalcoholic solvent extract of the water plantain is dried in the test pipes. After drying the hydroalcoholic solvent extract of water plantain, 2 cc of blood sample is added into test pipes. The blood clotting time is measured in all the test pipes. The test pipe 5 is control pipe, as neither hydroalcoholic solvent extract of water plantain nor the powder of the water plantain plant is added in the test pipe. The test pipe 6 comprises the water plantain plant powder and blood sample. Table 1 which is given below illustrate the result obtained after testing the blood coagulation property of the hydroalcoholic solvent extract of water plantain plant and the powder of the water plantain plant:

| | | | | Control | Test |
|---|---|---|---|---|---|
| Test Pipe 1 | Test Pipe 2 | Test Pipe 3 | Test Pipe 4 | Pipe 5 | Pipe 6 |
| 25 mg/L (λ) | 50 mg/L (λ) | 75 mg/L (λ) | 100 mg/L (λ) | Control | Powder |
| concentrated | concentrated | concentrated | concentrated | | |
| hydroalcoholic | hydroalcoholic | hydroalcoholic | hydroalcoholic | | |
| solvent extract | solvent extract | solvent extract | solvent extract | | |
| Clotting Time- | Clotting Time- | Clotting Time- | Clotting Time | Clotting | Clotting |
| 4 minute | 4 minute | 4 minute | 4 minute | Time | Time |
| 15 seconds | 30 seconds | 33 seconds | 43 seconds | 4 Minutes | 8 Minutes |
| | | | | | 7 Seconds |

According to one embodiment herein, the Table 1 results illustrate that the test pipe 1 comprising less water plantain hydro alcoholic solvent extract takes less time for clotting blood sample when compared to the that of the water plantain powder extracts with a plurality of mutually different concentration levels.

According to one embodiment herein, the ointment formulation comprises a predetermined amount of hydro alcoholic solvent extract of water plantain and a predetermined amount of Vaseline.

According to one embodiment herein, the ointment comprising hydro alcoholic solvent extract of water plantain and Vaseline is tested for treating burns and bleeding in rats. The skin wounds are created on the skin of a plurality of rats. The rats are anesthetized by intraperitoneal injection of ketamine xylazine.

The rat's nape is cleaned with a razor. A shape “O” is taken from the rat skin to get a plurality of wounded regions. The topical treatment of the skin wound is initiated on the first day. The topical treatment comprises application of the ointment comprising hydro alcoholic solvent extract of water plantain and Vaseline. The ointment comprising hydro alcoholic solvent extract of water plantain and Vaseline is a semisolid. The semisolid ointment comprising hydro alcoholic solvent extract of water plantain and Vaseline is applied on the wounds using swaps twice a day. The day of creating a rat skin wound and starting the topical treatment is “zero-day”. During the different stages of topical treatment, the compliance with ethical issues is maintained and any form of physical abuse and non-essential methods to the rats are avoided. On the rat's body, a wound is considered as control wound. The control wound does not receive any topical treatment. The topical treatment of the rat skin wound is continued for two weeks. On the third day, the wound receiving the topical treatment illustrated a reduction in the diameter of the wound. On fourteenth day, a complete recovery of the wound receiving the topical treatment with the ointment comprising hydro alcoholic solvent extract of water plantain and Vaseline is observed.

According to one embodiment herein, the most effective nutrients/elements present in the water plantain solvent extract stops bleeding and promote healing of tissues. The nutrients/elements of water plantain solvent extract which stops bleeding and promote healing of tissues include resin, fatty acids, palmitic acid and stearic acid. The resin heals the wound faster. The resin heals the wound faster as the resin has adherence properties for the treatment of wounds and burns. The starch and the carbohydrate present in the water plantain solvent extract act a growth factor for tissue.

According to one embodiment herein, the effect and the interaction of the ointment comprising hydro alcoholic solvent extract of water plantain plant and Vaseline with metabolic growth factor is also analyzed. The role of hydro alcoholic solvent extract on the gene expression and protein expression of growth factors analyzed. The growth factors analyzed are basic fibroblast growth factor (bFGF), transforming growth factor beta (TGFB) and vascular endothelial growth factor (VEGF).

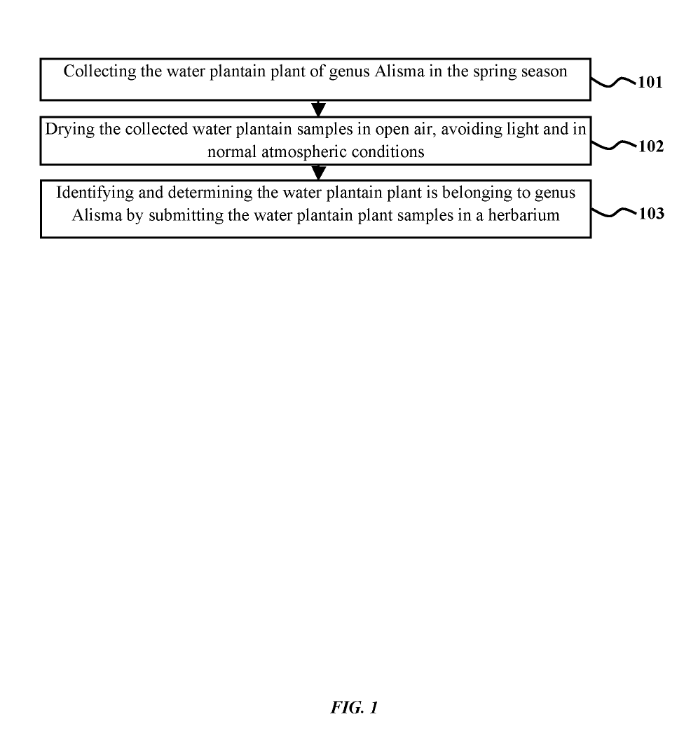

FIG. 1 illustrates a flowchart indicating a method for pre-treatment of the water plantain plant samples, according to one embodiment herein. The water plantain plant of genus Alisma in the spring season is collected (101). The collected water plantain samples is dried in open air, avoiding light and in normal atmospheric conditions (102). The water plantain plant is identified and determined for belonging to genus Alisma by submitting the water plantain plant samples in a herbarium (103).

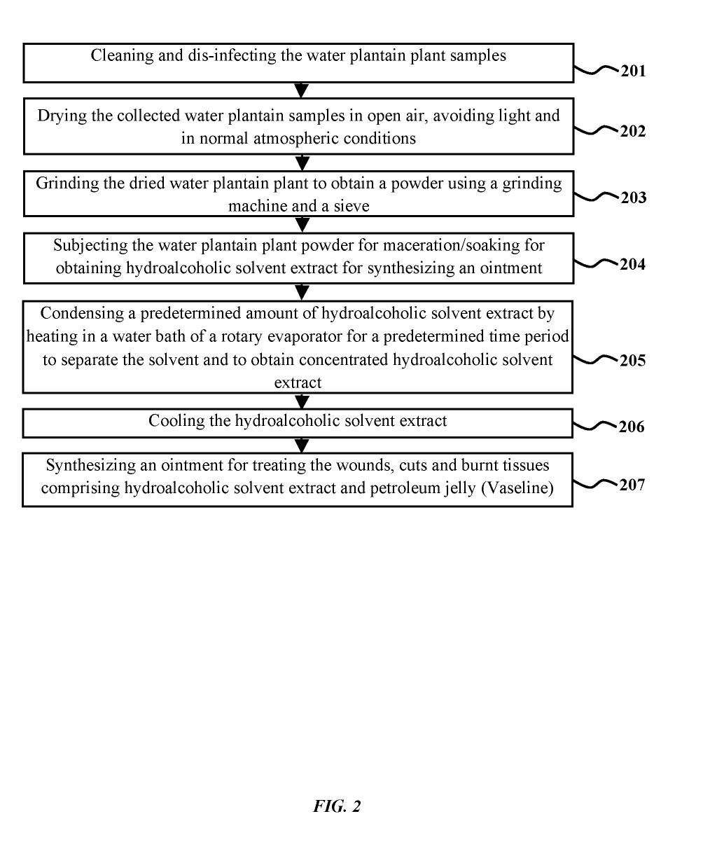

FIG. 2 illustrates a flowchart explaining a method for extracting the hydro alcoholic solvent extract comprising the essential nutrients and components of water plantain plant for synthesizing an ointment for treating the wounds, cuts and burnt tissues, according to one embodiment herein. The water plantain plant sample is cleaned and dis-infected (201). The collected water plantain samples are dried in open air, avoiding light and in normal atmospheric conditions (202). The dried water plantain plant is ground to obtain a powder using a grinding machine and a sieve (203). The water plantain plant powder is subjected for maceration/soaking for obtaining hydro alcoholic extract for synthesizing an ointment (204). A predetermined amount of hydro alcoholic solvent extract is condensed by heating in water bath of a rotary evaporator for a predetermined time period to separate the solvent and to obtain concentrated hydro alcoholic solvent extract (205). The hydro alcoholic solvent extract is cooled (206). An ointment for treating the wounds, cuts and burnt tissues is synthesized comprising hydro alcoholic solvent extract and petroleum jelly (Vaseline) (207).

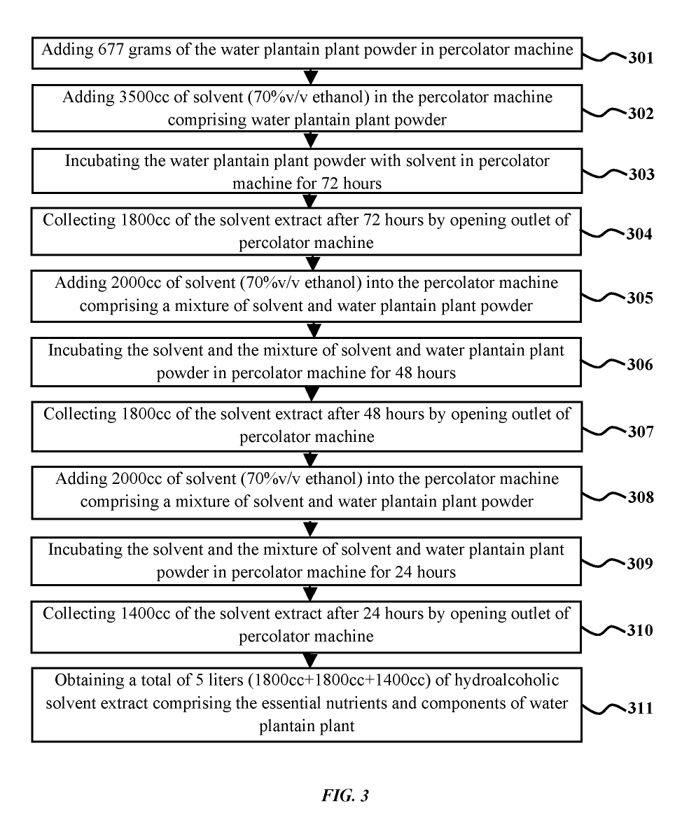

FIG. 3 illustrates a flow chart explaining a method for maceration/soaking for obtaining hydro alcoholic solvent extract for synthesizing an ointment, according to one embodiment herein. 677 grains of water plantain plant powder is added in percolator machine (301). 3500 cc of solvent (70% v/v ethanol is added in the percolator machine comprising water plantain plant powder (302). The water plantain plant powder is incubated with solvent in the percolator machine for 72 hours (303). 1800 cc of the solvent extract is collected after 72 hours by opening n outlet of the percolator machine (304). 2000 cc of solvent (70% v/v ethanol) is added into the percolator machine comprising a mixture of solvent and water plantain plant powder (305). The solvent, the mixture of solvent and water plantain plant powder are incubated in percolator machine for 48 hours (306). 1800 cc of the solvent extract is collected after 48 hours by opening the outlet of percolator machine (307). 2000 cc of solvent (70% v/v ethanol) is added into the percolator machine comprising a mixture of solvent and water plantain plant powder (308). The solvent, the mixture of solvent and water plantain plant powder are incubated in percolator machine for 24 hours (309). 1400 cc of the solvent extract is collected after 24 hours by opening outlet of the percolator machine (310). A total of 5 liters (1800 cc+1800 cc+1400 cc) of hydro alcoholic solvent extract is obtained comprising essential nutrients and components of water plantain plant (311).

It is to be understood that the phraseology or terminology employed herein is for description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A method for extracting the hydro alcoholic solvent extract comprising nutrients and components of water plantain plant for synthesizing an ointment for application on wounds, cuts and burnt tissues, the method comprises the steps of:
pre-treating a plurality of water plantain plant samples;
cleaning and dis-infecting the plurality of water plantain plant samples;
drying the plurality of cleaned and disinfected water plantain plant samples in open air and in normal atmospheric conditions, and wherein the plurality of water plantain plant samples are dried in a shade area or dark room without exposing to any light source;
grinding the dried water plantain plant samples to obtain a powder using a grinding machine and a sieve;
subjecting the water plantain plant powder for maceration/soaking in a solvent for obtaining hydro alcoholic extract for synthesizing an ointment;
condensing a predetermined amount of hydro alcoholic solvent extract by heating in a water bath of a rotary evaporator for a predetermined time period, and wherein the hydro alcoholic solvent extract is heated to separate the solvent and to obtain a concentrated hydro alcoholic solvent extract;
cooling the concentrated hydro alcoholic solvent extract; and
analysing a plurality of nutrients and components present in the hydro alcoholic solvent extract obtained from water plantain by Gas chromatography-mass spectrometry (GC-MS) machine, and wherein the hydro alcoholic solvent extract obtained from the water plantain plant and the powder of the water plantain plant are analysed for blood clotting blood/blood coagulating properties.

2. The method according to claim 1, wherein the step of pre-treating the water plantain plant sample comprises:
collecting the water plantain plant samples of genus Alisma in the spring season;
drying the collected water plantain plant samples in open air, avoiding light and in normal atmospheric conditions; and
determining whether the collected water plantain plant samples belong to genus Alisma by testing the water plantain plant samples in a herbarium, and wherein the collected water plantain plant samples are confirmed to belong to genus Alisma.

3. The method according to claim 1, wherein the step of subjecting the water plantain plant powder for maceration/soaking in a solvent for obtaining hydro alcoholic extract for synthesizing an ointment, comprises:
adding a preset amount of the water plantain plant powder in percolator machine, and wherein the preset amount of the water plantain plant powder added to the percolator machine 677 grains;
adding preset amount of solvent in the percolator machine comprising water plantain plant powder, and wherein the solvent is 70% v/v ethanol, and wherein the preset amount of the solvent added to the percolator machine 3500 cc;
incubating the water plantain plant powder with solvent in percolator machine for a predetermined time period, and wherein the predetermined time period is 72 hours;
collecting a preset amount of the solvent extract after 72 hours by opening an outlet of the percolator machine, and wherein the preset amount of the solvent extract collected after 72 hours through the opening outlet of percolator machine is1800 cc;
adding a preset amount of solvent into the percolator machine comprising a mixture of solvent and water plantain plant powder, and wherein the solvent is the 70% v/v ethanol, and wherein the preset amount of solvent added to the percolator machine comprising a mixture of solvent and water plantain plant powder is 2000 cc;
incubating the solvent and the mixture of solvent and water plantain plant powder in percolator machine for a predetermined time period, and wherein the predetermined time period is 48 hours;
collecting a preset amount of the solvent extract after 48 hours by opening the outlet of percolator machine, and wherein the preset amount of solvent extract collected after 48 hours by opening the outlet of percolator machine is 1800 cc;
adding a preset amount of solvent into the percolator machine comprising a mixture of solvent and water plantain plant powder, and wherein the solvent is the 70% v/v ethanol, and wherein the preset amount of solvent added to the percolator machine is 2000 cc;
incubating the solvent and the mixture of solvent and water plantain plant powder in percolator machine for a predetermined period of time, and wherein the predetermined time period is 24 hours;
collecting a preset amount of the solvent extract after 24 hours by opening the outlet of percolator machine, and wherein the preset amount of the solvent extract collected after 24 hours by opening the outlet of percolator machine is1400 cc;
obtaining a preset total amount of hydro alcoholic solvent extract comprising the essential nutrients and natural components of water plantain plant, and wherein the total amount of a hydro alcoholic solvent extract total of 5 litres (1800 cc+1800 cc+1400 cc).

4. The method according to claim 1, wherein the Gas chromatography-mass spectrometry (GC-MS) analysis of hydroalcoholic solvent extract obtained from the plurality of water plantain samples illustrates a presence of a plurality of nutrients and components, and wherein the plurality of nutrients and components are proteins, starch, resins, furfur aldehyde, asparagine, alkaloid, sugar, phytosterol, a plurality of fatty acids and a plurality of colour less solids.

5. The method according to claim 4, wherein the plurality of fatty acids are palmitic acid, stearic acid, linoleic acid and oleic acid.

6. The method according to claim 4, wherein the resin component in the hydro alcoholic solvent extract is a tissue regeneration agent, and wherein the resin provides nutrients required for a cell division and wherein the resin promotes a tissue healing process.

7. The method according to claim 1, wherein hydro alcoholic solvent extract at a plurality of concentration levels are tested for blood clotting/blood coagulation property, and wherein the plurality of concentration levels of the hydro alcohol solvent extract are 25 mg/L (λ), 50 mg/L (λ), 75 mg/L (λ) and 100 mg/L (λ), and wherein the hydro alcohol solvent extract with a concentration of 25 mg/L (λ) illustrate a time period of 4 minute 15 seconds for clotting 2 cc of blood when compared to the hydro alcohol solvent extract with a mutually different concentration levels, and wherein the mutually different concentration levels are 50 mg/L (λ), 75 mg/L (λ) and 100 mg/L (λ).

8. A composition for application on wounds, cuts and burnt tissues, the composition comprises:
a predetermined amount of hydroalcoholic solvent extract, and wherein the hydroalcoholic solvent extract is obtained from the water plantain plant powder by a maceration/soaking method; and
a predetermined amount of a petroleum jelly, and wherein the petroleum jelly is Vaseline, and wherein a plurality of nutrients and components are present in the hydro alcoholic solvent extract obtained from water plantain plant.

9. The composition according to claim 8, wherein the plurality of nutrients and components present in the hydro alcoholic solvent extract are a plurality of proteins, starch, resins, furfur aldehyde, asparagine, alkaloid, sugar, phytosterol, a plurality of fatty acids and a plurality of colour less solids.

10. The composition according to claim 9, wherein the plurality of fatty acids are palmitic acid, stearic acid, linoleic acid and oleic acid.

11. The composition according to claim 9, wherein the resin component in the hydro alcoholic solvent extract is a tissue regeneration agent, and wherein the resin provides nutrients required for a cell division and wherein the resin promotes tissue healing.
